(19) United States
(12) Patent Application Publication
(10) Pub. No.: US 2011/0053225 A1
(43) Pub. Date: Mar. 3, 2011

(54) MULTIFUNCTIONAL TAGS

(75) Inventors: Kristian Becker, Malmo (SE); Leif Bulow, Lund (SE); Ake Danielsson, Uppsala (SE); Peter Lindahl, Uppsala (SE); James Van Alstine, Uppsala (SE)

(73) Assignee: GE HEALTHCARE BIO-SCIENCES AB, UPPSALA (SE)

(21) Appl. No.: 12/863,284

(22) PCT Filed: Jan. 23, 2009
(86) PCT No.: PCT/SE09/00029

§ 371 Date: Jul. 16, 2010

(30) Foreign Application Priority Data
Jan. 24, 2008 (SE) 0800174-5

(51) Int. Cl. C12P 21/00; C07K 7/06; C12N 15/63

(52) U.S. Cl. 435/71.1; 530/329; 435/320.1; 530/328

(57) ABSTRACT

The present invention relates to multifunctional protein production tags comprising at least 4, preferably 4-8 amino acids, for optimising expression of proteins and purification thereof by various multi-step processes including chromatographic or filtration, as well as batch unit operations. The tags include sequences of multiple defined purposes which are not generated in defined linear sequence regions related to one purpose but as integrated sequences often overlapping each other, i.e. the defined purposes are not discrete separate units on the tag but rather heterogeneously distributed in the tag. The invention also relates to an expression vector encoding the multipurpose tag and a method for protein purification, comprising expressing a protein in the expression vector and purifying the protein in several steps using functionalities of the multipurpose tag.

### CROSS-REFERENCE TO RELATED APPLICATIONS

This application is a filing under 35 U.S.C. §371 and claims priority to international patent application number PCT/SE2009/000029 filed Jan. 23, 2009, published on Jul. 30, 2009, as WO 2009/093962, which claims priority to patent application number 0800174-5 filed in Sweden on Jan. 24, 2008.

### FIELD OF THE INVENTION

The present invention relates to multifunctional biomolecule tags for optimising expression of proteins and purification thereof by various multi-step processes including chromatographic or filtration, as well as batch unit operations. The tags include sequences of multiple defined purposes which are not generated in defined linear sequence regions related to one purpose but as integrated sequences often overlapping each other, i.e. the defined purposes are not discrete separate units on the tag but rather heterogeneously distributed in the tag.

### BACKGROUND OF THE INVENTION

Today the majority of proteins are produced by recombinant techniques and this has led to an increase of both the scale of separation processes (fermentation volumes) and the crude ferment concentration of target proteins. This makes volume reducing, high capacity techniques (e.g. partition or affinity methods) particularly attractive. Recombinant tags have proven very useful for protein purification. One of the most common tags contains six histidines (6His) for use in immobilised metal affinity chromatography (IMAC) and related separation methods (J. Porath, Metal chelate affinity chromatography, a new approach to protein purification, Nature 258, 598-599, 1975).

EP 0184355B1 describes tags containing two to five amino acids. These tags are suitable for IMAC. These include tags containing at least one histidine plus other “electron rich” amino acids such as lysine, methionine, valine, phenylalanine, tyrosine and tryptophan (lys, met, val, phe, tyr and trp). As noted in the patent these amino acids are included to improve the function of the tags, i.e. IMAC based purification.

It has previously been shown that proteins modified with terminal tags rich in aromatic residues exhibit increased partition into the less polar phase in APTPS, aqueous polymer two-phase systems (K. Köhler, C. Ljungquist, A. Kondo, A., Veide, B. Nilsson, Engineering proteins to enhance their partition coefficients in aqueous two-phase systems, Bio/technology, vol. 9, pp. 642-646, 1991). Such phases typically contain poly(ethylene glycol) [i.e., PEG] or other “ethoxy-rich” polymers.

In some cases proteins may be produced with two separate tags to enhance purification and use. Such tags may be localised either at two different physical locations in the protein, or in the form of a dual tag localised at one location. Dual tags will typically involve two separate recognisable regions of amino acid residue sequences which provide for the two separate functions. Following expression, such tags may be used for two or more tag dependent separation steps which may result in a more pure protein compared to only one such step.

However, even if dual tags are useful in many cases, it is often desired to keep the tag size as low as possible because larger tags may disturb the expression and function of the desired proteins; as well as the function of the tags. Such considerations limit the number of “desired functionalities” that can be incorporated as specific defined regions into tags—even though in many applications including larger scale separations it would be beneficial to have tags with multiple functionalities enabling more than two protein separations or other applications based on using the same tag. Therefore, there is a need to develop multi-tags which offer multi-functionalities and relatively smaller sized tag regions.

### SUMMARY OF THE INVENTION

The present invention relates to small multipurpose tags having several functionalities which are heterogeneously integrated in the tag, i.e. not disposed as discrete units in the tag as in prior art.

Thus, the invention relates to use of multipurpose tags which are a mixture of different functionalities which leads to smaller tags, compared to prior art tags built of discrete units each having a separate functionality. Other benefits of the multitags of the invention are that within practical limitations they do not negatively impact the expression and function of the desired proteins, as might be expected in some cases from larger tags made up by combining various linear sequences of various tags each added to fulfill a defined function.

In a first aspect, the invention relates to a multipurpose tag for tagging a biomolecule comprising integrated functionalities enhancing protein recovery, wherein said integrated functionalities comprise at least 4, preferably 4-8 amino acids, and are heterogeneously distributed in the tag. The integrated functionalities may at least partially overlap each other. The biomolecule may be a protein, polypeptide, nucleic acid, lipid, carbohydrate, polysaccharide or natural or otherwise produced conjugates of such biomolecules including glycoproteins and recombinant fusion proteins.

The multipurpose tag may be either created or added to protein or other target following expression via synthetic, catalytic, enzymatic or other (e.g. spontaneous deamidation or carboxylation) routes.

Naturally a protein might contain more than one multitag wherein said tags could be similar or different in structure and function and localised at different regions of the protein.

The functionalities of the multipurpose tag may be selected from affinity, hydrophobic interaction (HI), ion exchange (IE), multi-modal (MM) aqueous two phase separation (APTPS) partition, covalent extraction, precipitation, flocculation and filtration. Preferably, the multipurpose tag comprises at least three functionalities. The functionalities may be performed in, for example, bead or membrane format. A preferred affinity functionality is immobilized metal affinity chromatography, IMAC. In this case, the multipurpose tag preferably comprises at least two His-residues.

In a preferred embodiment, a combined HI and IE functionality is obtained from the hydrophobic and charged amino acids Glu, Lys, Arg, Phe, Asp, Trp and/or Tyr.

In the most preferred embodiment, tag comprises six amino acids including two H, preferably two non-adjacent H. In this embodiment, preferably the four other amino acids are selected from Glu, Lys, Arg, Phe, Asp, Trp and/or Tyr.

When the functionalities comprise HIC and/or APTPS partition, the tag comprises one or more hydrophobic amino acid residues. When the functionalities comprise IE, the tag comprises charged amino acids.

In a second aspect, the invention relates to a vector encoding a multipurpose tag as described above and a biomolecule. Optionally the vector comprises a standard tag, a multiple cloning site and a cleavage site for cleaving the tag from the biomolecule.

In a third aspect the invention relates to a method for protein purification, comprising expressing a protein in the above expression vector and purifying the protein in several steps using at least three functionalities of the multipurpose tag according to the invention.

Preferably the purification steps comprise APTPS, affinity, such as IMAC, IEC and HIC, and mixed mode formats using one and the same multipurpose tag. Purification steps not including the multipurpose tag may also be included, such as size exclusion chromatography. More than one multitag may be used; either at the same or different sites on a biomolecule.

### BRIEF DESCRIPTION OF THE DRAWINGS

FIG. 1 is a graph showing a direct relationship between the log kD of interaction between the tag and a test IMAC ligand and the relative electrophoretic migration of the tagged proteins.

FIG. 2 shows IEC (Ion Exchange Chromatography) evaluation of the GFP library of Example 1.

FIG. 3 shows IMAC evaluation of the GFP library.

FIG. 4 shows APTPS (Aqueous Two Phase System) evaluation of the GFP library.

FIG. 5 shows evaluation of tagged LDH according to Example 2 on IEC and IMAC.

FIG. 6 shows evaluation of tagged HHb according to Example 3 on IEX, IMAC and APTPS.

### DETAILED DESCRIPTION OF THE INVENTION

The present inventors provide novel multitags where separate functions do not have to involve separate sequence regions, but can involve integrated or overlapping sequences involving residues chosen to offer multiple effects. This results in shorter tags which can offer less negative impact on protein expression and other features. A benefit of the invention is that in some cases target protein expression is even enhanced by such “heterogenic” tags. Note that some of the aromatic and basic amino acids which confer enhanced separation performance may also act to enhance specific protease activity. This suggests that the multipurpose tags may be provided with integrated cleavage sites for removal of the tag after expression and purification of the tagged proteins.

A preferred recovery method according to the invention comprises four steps based on complementary interactions. Thus chromatography might be replaced by capture based filtration or batch methods including precipitation:
I. APTPS partition of crude fermentation broth—to reduce volumes and concentrate target protein expressed with multifunctional tag. Such steps also typically reduce nucleic acid, endotoxin, cell debris and other (column fouling and product affecting) contaminants which tend to favour the more polar phase.
II. IMAC to purify and further concentrate tagged target protein
III. An orthogonal purification step, such as HIC, to purify target protein (e.g. resolving active and non-active tagged protein). In this step HIC might be replaced or used in addition to ion-exchange chromatography (IEC) or multimodal (HIC/IEC) step.
IV. Polishing

The inventors have found that IMAC tags with fewer histidine residues and possibly tryptophan residues replaced with tyrosine residues may be easier to incorporate into some proteins via some expression systems.

Furthermore, partition and HIC effects are sensitive to histidine groups being charged in a pH dependent manner. This led the inventors to conclude that reducing the number of his-groups can aid separations based on HIC or partition.

The present inventors have investigated HIC interactions involving tagged green fluorescent proteins (GFPs) and have optimised tag constructs for HIC and IMAC. In a preferred embodiment, the present invention relates to various multi-step processes involving IMAC, HIC and two-phase partition. In table 1, some possible multitags for GFP purification are listed (in standard one letter format) as is 6 His type tag for comparison purposes.

TABLE 1

| | | | | | | Cu(II)- |
|---|---|---|---|---|---|---|
| | | | | | IMAGE | VBIDA |
| | | Total | Seq'l. | | Migration | KD (μM) |
| Clone | Tag Sequence⁴ | Tag His | His* | pI | (relative) | (r² ≈ 0.98) |
| n-GFP | MEFELGT | 0 | 0 | 5.57 | 0.85 | 1680 |
| | (SEQ ID NO: 1) | | | | | |
| GFP2 | MEFHSVGMH | 2 | 1 | 5.82 | 0.86 | 1200 |
| | SEQ ID NO: 3) | | | | | |
| GFP8 | MEFHGGAEH | 2 | 1 | 5.72 | 0.69 | |
| | (SEQ ID NO: 4) | | | | | |
| GFP9 | MEFHDMMAH | 2 | 1 | 5.72 | 0.67 | |
| | (SEQ ID NO: 5) | | | | | |
| GFP12 | MEFHSVGLH | 2 | 1 | 5.82 | 0.57 | 200 |
| | (SEQ ID NO: 6) | | | | | |
| GFP24 | MEFHLRARH | 2 | 1 | 6.05 | 0.44 | 150 |
| | (SEQ ID NO: 7) | | | | | |
| GFP25 | MEFHRPMRH | 2 | 1 | 6.05 | 0.39 | 140 |
| | (SEQ ID NO: 8) | | | | | |
| GFP26 | MEFHWRSRH | 2 | 1 | 6.05 | 0.39 | |
| | (SEQ ID NO: 9) | | | | | |
| GFP27 | MEFHWVARH | 2 | 1 | 5.94 | 0.38 | 120 |
| | (SEQ ID NO: 10) | | | | | |
| GFP28 | MEFHITGHH | 3 | 2 | 5.88 | 0.35 | 110 |
| | (SEQ ID NO: 11) | | | | | |
| GFP29 | MEFHHSLVH | 3 | 2 | 5.88 | 0.28 | 100 |
| | (SEQ ID NO: 12) | | | | | |
| GFP30 | MEFHAHHLH | 4 | 3 | 5.94 | 0.21 | 90 |
| | (SEQ ID NO: 13) | | | | | |
| 6His-GFP | MGHHHHHHGT | 6 | 6 | 6.13 | 0.14 | 70 |
| | (SEQ ID NO: 14) | | | | | |

Notes
1. pI refers to isoelectric pH
2. IMAGE refers to immobilised metal affinity gel electrophoresiswhere migration increases inversely to
affinity. So the lower the number the greater the affinity.
3. Cu VIBDA (VBIDA stands for N-(4-vinyl)-benzyl iminodiacetic acid) refers to affinity of metal ion for
the tag and is given as dissociation constant KD in micromolar concentration. As with IMAGE the lower
the number the greater the affinity.

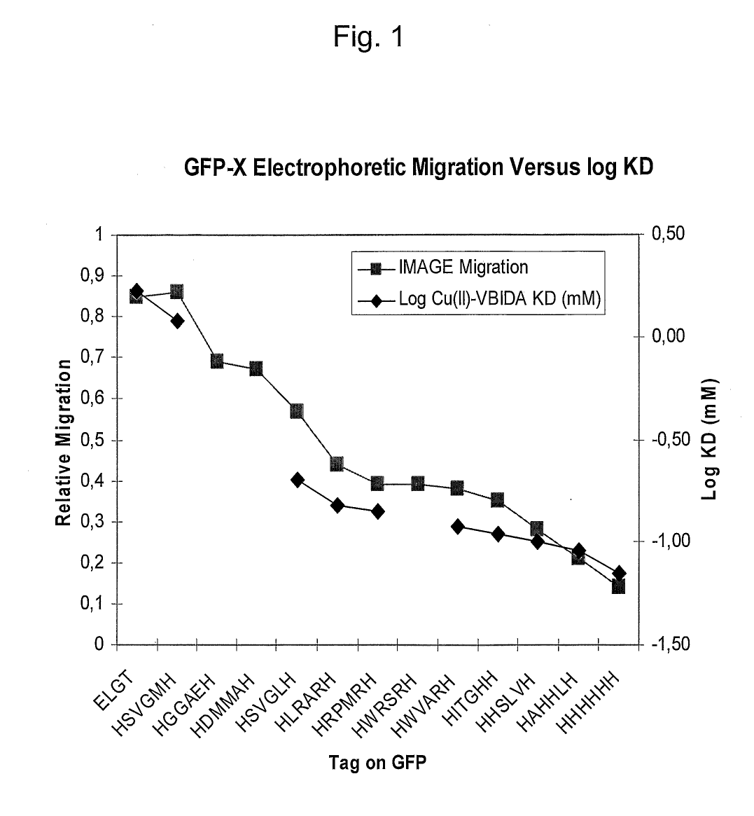

FIG. 1 shows a graph of a direct relationship between the log kD of interaction between the tags and a test IMAC ligand (VBIDA) and the relative electrophoretic migration of the tagged proteins. In this figure the kD data is given in millimolar not micromolar units.

A tag such as HisAlaHisHisLeuHis (GFP30) has similar affinity (kD 90 μM) to the conventional 6His (70 μM) while tags such as those on GFP25 to 27 offer suitably to low kD values (≦120 μM) to be as useful as standard 6His tags for IMAC. These values are suitably low for such tags to be used in a separation process with binding of target at high enough affinity to allow for ease of washing and recovery.

Tags such as 25 and 27 further offer significant increases in interaction of the proteins with HIC media as well as other tags in the Table, such as those rich in tyrosine. Such tags may offer increased expression of target protein over that of the 6His alternative. The ability of some of the tags to alter protein pI should also improve use of such tagged proteins with other separation and analytical methods such as those involving ion exchange or mixed mode ion exchange plus hydrophobic interactions. Tags comprising aromatic residues can also participate in other interactions such as van der Waals and ion-pi interactions.

### EXAMPLES

Below the present invention will be disclosed by way of examples, which are intended solely for illustrative purposes and should not be construed as limiting the present invention as defined in the appended claims. All references mentioned below or elsewhere in the present application are hereby included by reference.

A tag containing several key properties has been cloned into the N-terminal of different model proteins. The proteins are: green fluorescent protein (GFP), lactate dehydrogenase (LDH) and human haemoglobin (HHb).

The chosen tag consists of the following amino acid sequence: HYDHYD (SEQ ID NO:20), i.e. two histidines (metal binding), two tyrosines (hydrophobic) and two aspartic acid (charged).

The separate properties of the amino acids of the tag have been evaluated using ion exchange chromatography (IEC), immobilized metal ion chromatography (IMAC) and aqueous two-phase systems (APTPS). Standard approaches were used and it is expected that more careful screening of conditions could enhance the effectiveness of each step.

The results show that proteins with the tag inserted have larger retention volumes on IEC, IMAC and HIC and partition more towards the hydrophobic phase in APTPS compared to the respective native proteins.

### Example 1

### GFP or Green Fluorescent Protein

A GFP library was constructed according to Table 2.

TABLE 2

| Tag | Property of tag | Amino acid sequence | Abbreviation |
|---|---|---|---|
| No tag | | MEF-GFP | GFP |
| Alanine tag | Neutral | MEFAAAAAA | A |
| | | (SEQ ID NO: 15)-GFP | |
| Histidine tag | Metal binding | MEFHAAHSA | HH |
| | | (SEQ ID NO: 16)-GFP | |
| Tyrosine/Histidine | Hydrophobic/metal | MEFHNAAYA | HY |
| tag | binding | (SEQ ID NO: 17)-GFP | |
| Aspartic acid tag | Charged | MEFPADAAD | DD |
| | | (SEQ ID NO: 18)-GFP | |
| Multifunctional tag | Metal binding/ | MEFHYDHYD | HYDHYD |
| | charged/hydrophobic | (SEQ ID NO: 19)-GFP | |

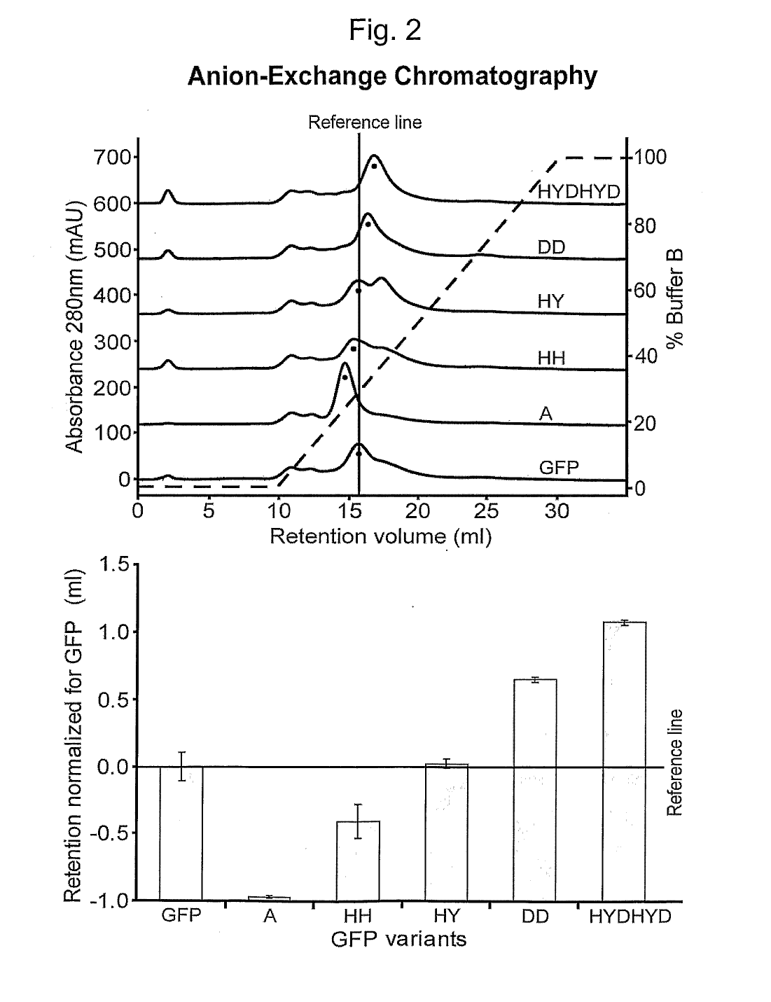

GFP was tagged at the N-terminal. Crude extracts were purified using heat-treatment 70 C, 10 min, salt precipitation 1.2-2.8M (NH₄)₂SO₄ and dialysis. MW for GFP=28 kDa.
FIG. 2 shows evaluation of the tag in IEC. As appears from the figure, the multitag and charged tags have clearly different retention volumes than the other tags. Note that column bed volume was one ml so that a peak shift of 1 ml represents considerable separation performance.
System: ÄKTAPURIFIER™

### Column: HITRAP™ (1 ml)

### Matrix: Q SEPHAROSE™

### Buffer A=20 mM Bis-Tris, pH 7.0

### Buffer B=20 mM Bis-Tris, 1M NaCl, pH 7.0

### Program:

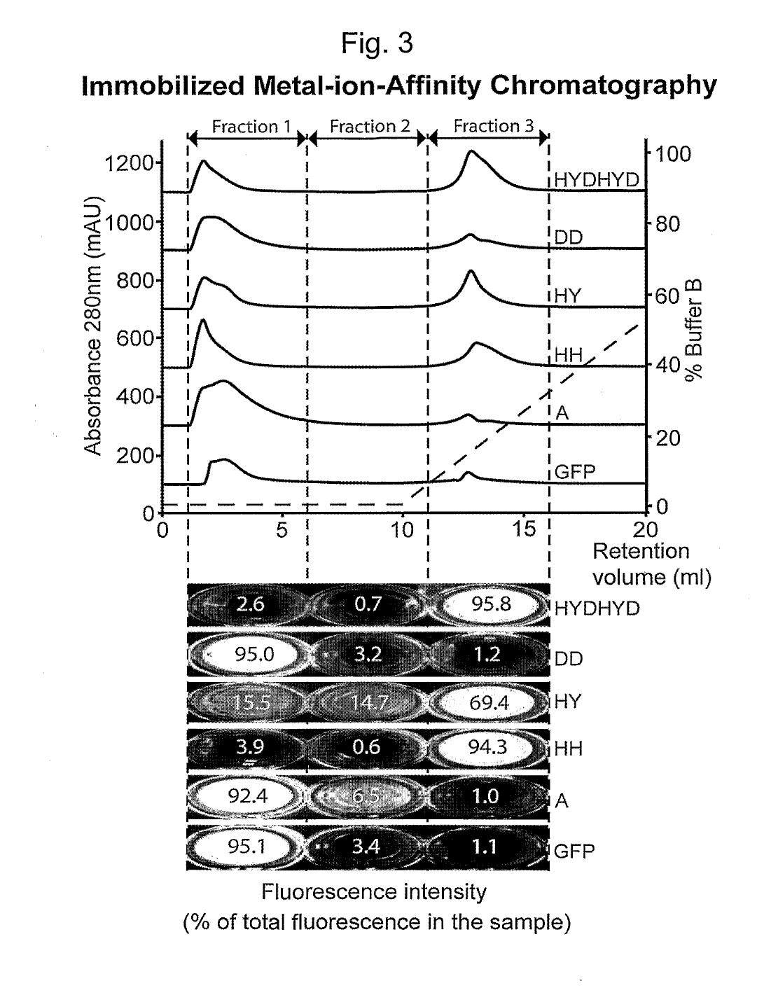

Flow=1 ml/min
Equilibration volume=12 CV
Gradient=20 CV (0-100% buffer B)
Fractions were collected and checked for fluorescence. Dot in chromatograms mark fluorescent peak. Reference line indicates retention volume of native GFP.
Histogram: three independent experiments.
FIG. 3 shows evaluation of the tag in IMAC. As appears from the figure, the multitag and metal binding tags bind to the column, the others do not.
System: ÄKTAPURIFIER™

### Column: HITRAP™ (1 ml)

### Matrix: Q SEPHAROSE™

### Buffer A=20 mM Bis-Tris, pH 7.0

### Buffer B=20 mM Bis-Tris, 1M NaCl, pH 7.0

### Program:

Flow=1 ml/min
Equilibration volume=12 CV
Gradient=20 CV (0-100% buffer B)

Fractions were collected and checked for fluorescence. Dot in chromatograms mark fluorescent peak. Reference line indicates retention volume of native GFP.

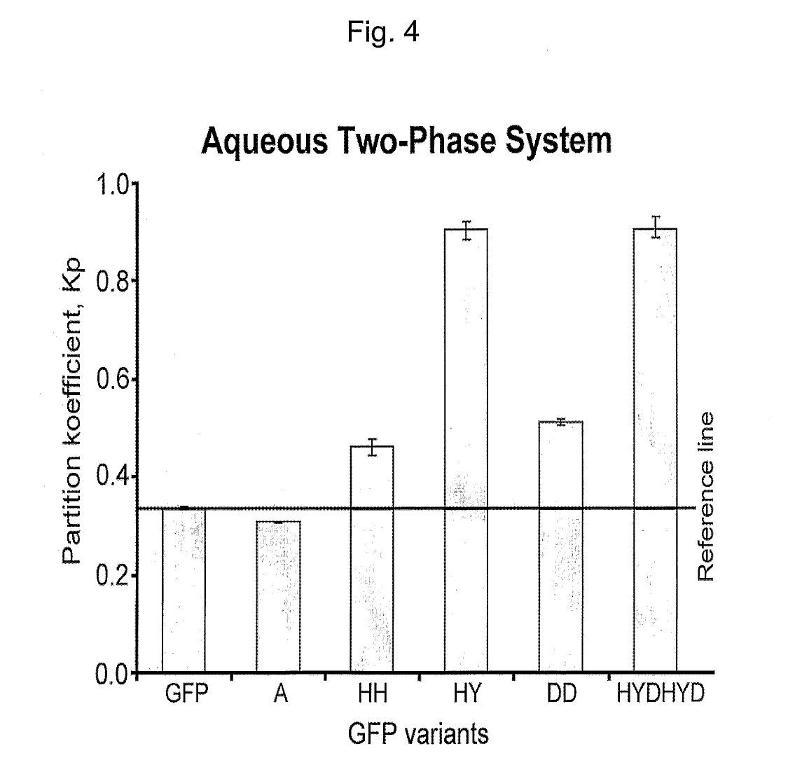

Histogram: three independent experiments.
FIG. 4 shows evaluation of the tag in APTPS. As appears from the figure, the multitag and hydrophobic tag are clearly more partitioned towards the PEG-rich upper phase than the other tags.
PEG/salt system:
10.3% (w/w) PEG4000
11.0% (w/w) K-phosphate, pH6.8 (HPO42-/H2PO4- mole ratio=1.42 (1.42-system)).
2 g system mixed with GFPs 2 min, rest 15 min, mix 2 min, centrifuge 10 min 800 g. Phases were separated and checked for fluorescence (compensated for background PEG/salt) Partition coefficient, Kp=fluorescence intensity in top phase/fluorescence intensity in bottom phase. Partition improvement, PI=Kp tag/Kp native. Reference line indicates partitioning of native GFP. PI is not to be confused with isoelectric pH (pI). Three independent systems.

### Example 2

### LDH or Lactate Dehydrogenase

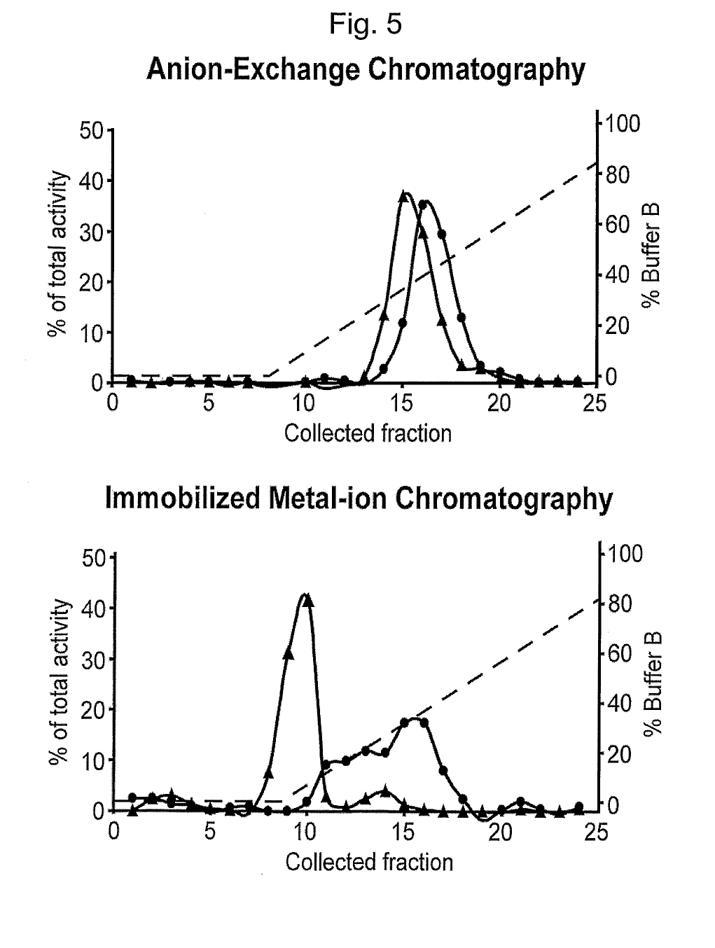

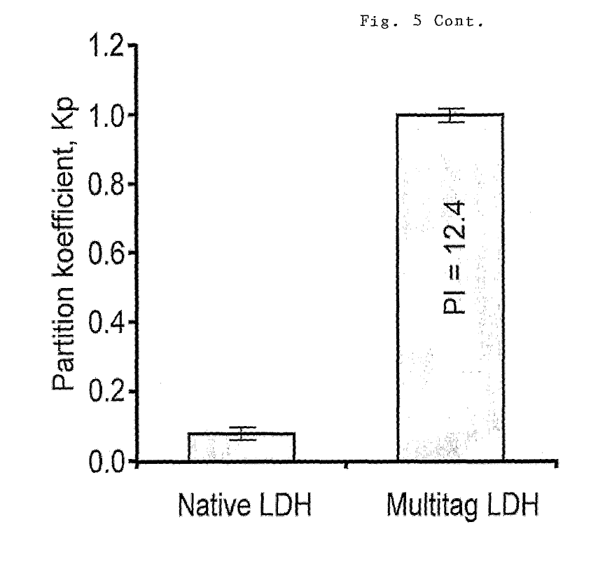

Homomeric tetramer lactate dehydrogenase (LDH) from Bacillus stearothermophilus was tagged with the previously described tag at the N-terminal. Mw for LDH was 34 kDa/subunit.
Construction MEF-LDH (LDH) and MEFHYDHYD(SEQ ID NO:19)-LDH (LDH with multifunctional tag).
Purified by heat treatment 70° C., 10 min.
FIG. 5 shows evaluation of LDH on IEC and IMAC. As appears from the figure, the multitag is functional also on LDH. Tagged LDH has longer retention on all chromatographic experiments and also portioned more towards the PEG phase in APTPS.
Experimental=same as for GFP experiments:
Partition coefficient, Kp=activity top phase/activity bottom phase. Partition improvement, PI=Kptag/Kpnative.
LDH activity was measured in 0.1M MES, pH 6.5, 30 mM pyruvate, 0.2 mM NADH and absorbance decrease was monitored at 340 nm.
LDH=triangles
HYDHYD(SEQ ID NO:20)-LDH=circles

### Example 3

### hHb or Human Hemoglobin

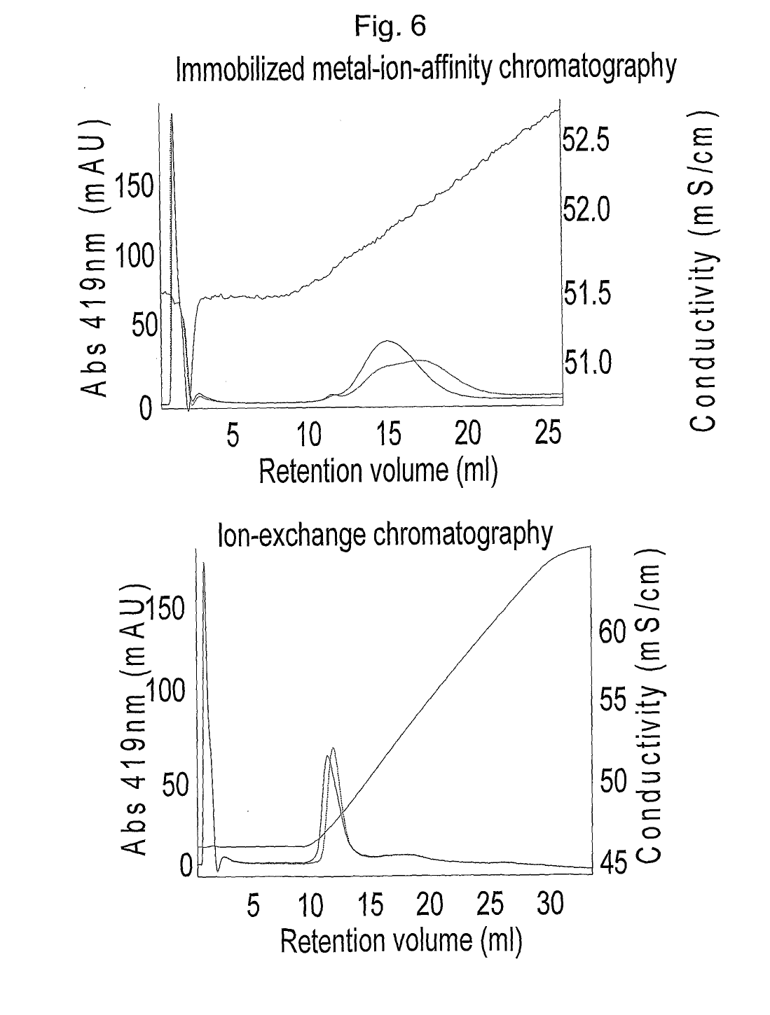

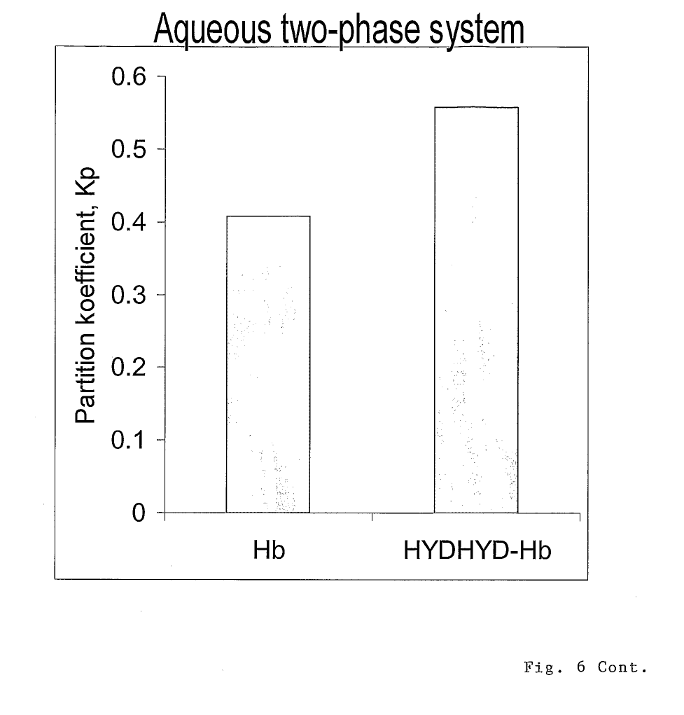

FIG. 6 shows evaluation of hHb on IMAC, IEC and APTPS.
As appears from the figure, tagged hHb improves the separation results.
Experimental=same as for GFP experiments:
Except for APTPS where pH is increases to pH 8.0.
Absorbance is measured at 419 nm (specific for CO—HHb)
hHb=solid line
HYDHYD(SEQ ID NO:20)-hHb=dotted line.

It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A multipurpose tag for tagging a biomolecule, comprising integrated functionalities enhancing protein production and recovery, wherein said integrated functionalities comprise at least 4, preferably 4-8 amino acids, and are heterogeneously distributed in the tag.

2. The multipurpose tag of claim 1, wherein the integrated functionalities are at least partially overlapping each other.

3. The multipurpose tag of claim 1, wherein the tag comprises at least three functionalities selected from affinity, hydrophobic interaction (HI), ion exchange (IE), multi-modal (MM) aqueous two phase separation (APTPS) partition, extraction, precipitation, flocculation and filtration.

4. The multipurpose tag of claim 3, wherein a combined HI and IE functionality is obtained from the hydrophobic and charged amino acids Glu, Lys, Arg, Phe, Asp, Trp and/or Tyr.

5. The multipurpose tag of claim 1, wherein the biomolecule is a protein, polypeptide, nucleic acid, lipid, carbohydrate, polysaccharide or natural or otherwise produced conjugates of such biomolecules including glycoproteins and recombinant fusion proteins.

6. The multipurpose tag of claim 3, wherein the affinity functionality is immobilized metal affinity chromatography, IMAC.

7. The multipurpose tag of claim 6, comprising at least two His-residues.

8. The multipurpose tag of claim 4, wherein the tag comprises six amino acids including two His.

9. (canceled)

10. The multipurpose tag of claim 3, wherein the functionalities comprise HI and/or APTPS partition, and the tag comprises one or more hydrophobic amino acid residues.

11. The multipurpose tag of claim 3, wherein the functionalities comprise IE and the tag comprises charged amino acids.

12. A vector encoding the multipurpose tag of claim 1, and optionally a standard tag and a cleavage site.

13. A method for protein purification, comprising expressing a protein in an expression vector which encodes the multipurpose tag of claim 1, and optionally a standard tag and a cleavage site and purifying the protein in several steps using at least three functionalities of the multipurpose tag of claim 1.

14. The method of claim 13, wherein the purification steps comprise APTPS, affinity, IE and HI.

15. The method of claim 13, further comprising one or more purification steps which do not use the functionalities of the multipurpose tag.

16. The method of claim 13, wherein more than one multitag is used; either at the same or different sites on a biomolecule.


SEQUENCE LISTING (20 sequences)

SEQ ID NO: 1 - protein, 7 aa, Artificial Sequence
  Synthetic peptide
MEFELGT

SEQ ID NO: 2 - protein, 9 aa, Artificial Sequence
  Synthetic peptide
MEFHXXXXH

SEQ ID NO: 3 - protein, 9 aa, Artificial Sequence
  Synthetic peptide
MEFHSVGMH

SEQ ID NO: 4 - protein, 9 aa, Artificial Sequence
  Synthetic peptide
MEFHGGAEH

SEQ ID NO: 5 - protein, 9 aa, Artificial Sequence
  Synthetic peptide
MEFHDMMAH

SEQ ID NO: 6 - protein, 9 aa, Artificial Sequence
  Synthetic peptide
MEFHSVGLH

SEQ ID NO: 7 - protein, 9 aa, Artificial Sequence
  Synthetic peptide
MEFHLRARH

SEQ ID NO: 8 - protein, 9 aa, Artificial Sequence
  Synthetic peptide
MEFHRPMRH

SEQ ID NO: 9 - protein, 9 aa, Artificial Sequence
  Synthetic peptide
MEFHWRSRH

SEQ ID NO: 10 - protein, 9 aa, Artificial Sequence
  Synthetic peptide
MEFHWVARH

SEQ ID NO: 11 - protein, 9 aa, Artificial Sequence
  Synthetic peptide
MEFHITGHH

SEQ ID NO: 12 - protein, 9 aa, Artificial Sequence
  Synthetic peptide
MEFHHSLVH

SEQ ID NO: 13 - protein, 9 aa, Artificial Sequence
  Synthetic peptide
MEFHAHHLH

SEQ ID NO: 14 - protein, 10 aa, Artificial Sequence
  Synthetic peptide
MGHHHHHHGT

SEQ ID NO: 15 - protein, 9 aa, Artificial Sequence
  Synthetic peptide
MEFAAAAAA

SEQ ID NO: 16 - protein, 9 aa, Artificial Sequence
  Synthetic peptide
MEFHAAHSA

SEQ ID NO: 17 - protein, 9 aa, Artificial Sequence
  Synthetic peptide
MEFHNAAYA

SEQ ID NO: 18 - protein, 9 aa, Artificial Sequence
  Synthetic peptide
MEFPADAAD

SEQ ID NO: 19 - protein, 9 aa, Artificial Sequence
  Synthetic peptide
MEFHYDHYD

SEQ ID NO: 20 - protein, 6 aa, Artificial Sequence
  Synthetic peptide
HYDHYD